(19) United States
(12) Patent Application Publication
(10) Pub. No.: US 2025/0108023 A1
(43) Pub. Date: Apr. 3, 2025

(54) Products for Masking Bitterness

(71) Applicants: The Third People's Hospital of Henan Province, Zhengzhou City (CN); Zhengzhou Weiqian Biotechnology Co., Ltd., Zhengzhou City (CN)

(72) Inventors: Mingkui Shen, Zhengzhou City (CN); Jingliang Zhang, Zhengzhou City (CN); Xiaoting Li, Zhengzhou City (CN); Hongna Cheng, Zhengzhou City (CN); Lili Jue, Zhengzhou City (CN); Chenmeng Gu, Zhengzhou City (CN); Linlin Jiao, Zhengzhou City (CN); Guangchong Zhang, Zhengzhou City (CN); Jiaxue Luo, Zhengzhou City (CN)

(73) Assignees: The Third People's Hospital of Henan Province, Zhengzhou City (CN); Zhengzhou Weiqian Biotechnology Co., Ltd., Zhengzhou City (CN)

(21) Appl. No.: 18/980,002

(22) Filed: Dec. 13, 2024

Related U.S. Application Data
(63) Continuation of application No. PCT/CN2023/112718, filed on Aug. 11, 2023.

(51) Int. Cl. A61K 31/192; A61K 31/05; A61K 31/09; A61K 31/11; A61K 31/122; A61K 31/197; A61K 31/4525

(52) U.S. Cl. CPC A61K 31/192; A61K 31/05; A61K 31/09; A61K 31/11; A61K 31/122; A61K 31/197; A61K 31/4525

(57) ABSTRACT

The present disclosure provides an application of TRPM8 agonist, TRPA1 agonist or TRPV1-4 agonist as a substance for masking bitterness, an application of one or more substances as a taste masking agent, an application of one or more substances in an oral spray or mouthwash for masking bitterness, and an oral spray or taste masking agent for masking bitterness. The present disclosure finds that TRPM8 receptor agonists and TRPV1-4 and TRPA1 agonists all have the effect of masking the perception of oral bitterness. TRPM8 receptor agonists stimulate cold channels, and TRPV1-4 and TRPA1 stimulate hot channels. The cool feeling can be used to neutralize the heat feeling, eliminating the uncomfortable and hot pain and bringing a relatively cool and pleasant pleasure.

### CROSS-REFERENCE TO RELATED APPLICATION

This application is a Continuation application of International Application No. PCT/CN2023/112718, filed on Aug. 11, 2023, entitled “Products for Masking Bitterness”, which claims priority to Chinese Application No. 202210659911.X, filed on Jun. 13, 2021, incorporated herein by reference in their entirety.

### TECHNICAL FIELD

The present disclosure relates to the field of daily necessities, food or medicine, in particular to products that are applied to oral cavities and designed for masking bitterness. With reference to such products, the present disclosure specifically relates to the use of TRPM8 agonist, TRPA1 agonist or TRPV1-4 agonist as a substance for masking bitterness, the use of one or more substances as a taste masking agent, the use of one or more substances in an oral spray or mouthwash for masking bitterness, and an oral spray or taste masking agent for masking bitterness.

### BACKGROUND

Human oral taste buds are composed of many taste cells, of which type I taste cells have glial-like properties and mainly act as supporting cells, while type II taste cells detect bitter, sweet and umami stimuli by activating GPCR pathways. This GPCR pathway consists of phospholipase C (PLC) signaling cascade that results in the release of calcium (Ca²⁺) from internal stores and subsequent activation of monovalent selective transient receptor potential melastatin 5 (TRPM5) channels. Type II cells lack traditional synaptic specializations and instead activate TRPM5 to cause cellular depolarization, which stimulates Calhm1 channels and leads to the release of ATP as a neurotransmitter. TRPM5 is unique compared to most TRP channels in that it is voltage-sensitive, monovalent cation-selective, and activated by the increasing intracellular Ca²⁺. Triphenylphosphine oxide (TPPO) is a reversible, selective TRPM5 channel blocker. Application of TPPO (5 μM) abolished the remaining taste-evoked Na⁺ responses in mice to which only TRPM5 is applied, indicating that these taste-evoked Na⁺ signals are mediated exclusively by TRPM5. Responses were restored following removal of TPPO. Further experiments found that both TRPM4 and TRPM5 are necessary and sufficient for the normal conduction of bitter, sweet, and umami stimuli. The main difference between TRPM4 and TRPM5 in other cell types is their sensitivity to intracellular Ca²⁺. Analysis in HEK-293 cells found that the Ca²⁺ concentration of half-maximal current activation (EC50) of TRPM4 (20.2±4.0 μM) was much greater than the EC50 of TRPM5 (0.70±0.1 μM). If these EC50 values are comparable in taste cells, we predict that TRPM5 will be activated first during cell stimulation when cytosolic Ca²⁺ levels are low. As Ca²⁺ concentrations increase, TRPM4 will also contribute to taste-evoked signals. These findings suggest that compared with TRPM4, TRPM5 requires a smaller increase in cytosolic Ca²⁺ to be activated. Because bitter, sweet, and umami stimulation signals follow the same pathway, sweetness and umami can mask bitterness through competition, for example, sucrose may mask bitterness. TRPM5 belongs to a family of transient receptor potential (TRP) channels. TRP channels are calcium-permeable non-selective cation channels. About 28 TRP channels have been identified, with different numbers of splice variants for each channel gene. TRP channels are divided into six different subgroups, including TRPV(1-6), TRPM(1-8), TRPC(1-7), TRPA1, TRPP(1-3), and TRPML(1-3), based on their sequence similarities. In general, TRP channels are involved in calcium handling (e.g., intracellular calcium mobilization and calcium reabsorption) and a wide range of sensory modalities, including pain, temperature, taste, etc. Abnormalities in TRP channels are part of the important mechanisms of several diseases, including neurodegenerative diseases, diabetes, inflammatory bowel disease, epilepsy, cancer, etc. Several members of the TRP family, TRPV1-4, TRPM8, and TRPA1, also known as “ThermoTRP”, are involved in detecting temperature changes, thereby acting as molecular thermometers of our body. In addition, they are multimodal nociceptors that integrate painful stimuli such as noxious temperature and chemical injury. For example, the TRPV1 channel mediates thermal hyperalgesia and pain induced by capsaicin and acid. TRPA1 is a nociceptor that integrates many noxious environmental stimuli, including oxidants and electrophiles. Recently, gene-deficient animals have been created to study the role of TRP channels in pain and nociception, and it has been confirmed that TRPV1, TRPV3, TRPV4, and TRPA1 are involved in nociception.

TRP channel protein sequences are highly similar, and the stimulation of many molecules will activate several protein channels at the same time, resulting in the simultaneous appearance of taste, temperature, and pain. For example, in the experiments conducted by the inventors, all six substances can act on the taste pathway TRPM5, thereby masking the taste channel, but each substance can also activate other pathways. For example, menthol can produce a cold sensation, clove oil produces a hot sensation, chili oil produces pain and heat sensation, and asarum produces a numb sensation.

### SUMMARY

The purpose of the present disclosure is to at least partially overcome the defects of the prior art, expand the raw material range of taste masking agents, provide a TRPM8 agonist, TRPA1 agonist or TRPV1-4 agonist as a substance for masking bitterness, the use of one or more substances as taste masking agents, the use of one or more substances in an oral spray or mouthwash for masking bitterness, and an oral spray or taste masking agent for masking bitterness.

The purpose of the present disclosure is further to provide one or more substances for use in an oral spray or mouthwash for masking bitterness and an oral spray or taste masking agent for masking bitterness, so that the effect of masking bitterness is better.

In order to achieve the above purposes or one of the purposes, the technical solutions of the present disclosure are as follow:

A use of TRPM8 agonist as a substance for masking bitterness, wherein the TRPM8 agonist comprises one or more of the following substances: menthol derivatives, citral, linalool, eugenol or eucalyptol;

wherein the menthol derivative comprises one or more of the following substances: menthyl amide, phenoxyethanol, limonene, 18-cineole, piperine, geranyl, menthyl acetate, WS-3 ((IR, 2S, 5R)-2-isopropyl-5-methyl-cyclohexanecarboxylic acid acetamide), WS-5 ([((IR, 2S, 5R)-2-isopropyl-5-methyl-cyclohexanecarbonyl)-amino]-ethyl acetate), WS-23 (2-isopropyl-2, 3, N-trimethyl-butyramide), menthyl lactate, menthyl succinate, menthoxypropanediol, 2-isopropyl-5-methylcyclohexyl 4-(dimethylamino)-4-oxobutyrate (2-isopropyl-5-methylcyclohexyl 4-(dimethylamino)-4-oxobutyrate).

According to an aspect of the present disclosure, there is provided a use of TRPA1 agonist as a substance for masking bitterness, wherein the TRPA1 agonist comprises one or more of the following substances:
cinnamaldehyde, mustard oil, allicin, capsaicin ester, carvacrol, eugenol, piperine, gingerol, wintergreen oil, clove oil, caryophyllene, caryophyllene oxide, citral or menthol.

According to an aspect of the present disclosure, there is provided a use of TRPV1-4 agonist as a substance for masking bitterness, wherein the TRPV1 agonist is selected from one or more of capsaicin, resiniferatoxin, vitamin D, piperine, evodiamine, scutellaria baicalenol, capsaicin ester, 6-shogaol, gingerol, gingerone, gingerol or polygonal.

According to an aspect of the present disclosure, there is provided a use of one or more substances as a taste masking agent, wherein the substances are selected from linalool, citral, cinnamaldehyde, caryophyllene, eugenol acetate, menthone, eucalyptus oil, borneol, menthamide, menthyl succinate, cooling agent WS-5, cooling agent WS-23, gingerol, piperine or allicin.

According to an aspect of the present disclosure, there is provided a use of one or more substances in an oral spray or mouthwash for masking bitterness, wherein:
the one or more substances are a Class A substance, a Class B substance, or a combination of Class A substance and Class B substance;
the Class A substance is one or more TRPM8 agonists; and the Class B substance is one or more of TRPA1 agonists or TRPV1-4 agonists.

According to some embodiments of the present disclosure, the one or more substances are the Class A substance, and a volume concentration of the Class A substance in the oral spray or mouthwash is 0.001%-0.5%; or
the one or more substances are the Class B substance, and a volume concentration of the Class B substance in the oral spray or mouthwash is 0.001%-0.5%; or
the one or more substances are a combination of Class A substance and Class B substance, and a volume concentration of the Class A substance in the oral spray or mouthwash is 0.001%-0.5%, and a volume concentration of the Class B substance in the oral spray or mouthwash is 0.001%-0.5%.

According to some embodiments of the present disclosure, the oral spray or mouthwash reaches a peak of the bitterness masking effect within 5-10 seconds after being sprayed in an oral cavity, and the effect lasts for 30-120 seconds.

According to an aspect of the present disclosure, there is provided an oral spray or taste masking agent for masking bitterness, wherein:
the oral spray or taste masking agent comprises a Class A substance, a Class B substance, or a combination of Class A substance and Class B substance;
the Class A substance is one or more of TRPM8 agonists; and the Class B substance is one or more of TRPA1 agonists or TRPV1-4 agonists.

According to some embodiments of the present disclosure, the oral spray or taste masking agent is used for drugs, and the drugs comprise any one of anti-tumor drugs, drugs for treating hyperlipidemia, drugs for treating hyperglycemia, analgesics, antibiotics, antipyretics or traditional Chinese medicines, and the dosage form of the drugs comprises any one of tablet, decoctions, dispersion, wine preparation, granule, mouthwash, liquid oral preparation or aerosol; or
the oral spray or taste masking agent is used for food, and the food comprises any one of ice cream, custard, cream, beverage or cake; or
the oral spray or taste masking agent is used for oral care products, and the oral care products comprise any one of toothpaste, mouthwash, chewing gum, breath freshener or mouthwash agent.

According to some embodiments of the present disclosure, the tablet comprises any one of a multilayer tablet, a chewable tablet, a soluble tablet, an effervescent tablet, an orodisintegrating tablet, a sustained-release tablet, a controlled-release tablet, a dispersible tablet, a lozenge, a sublingual tablet or an oral patch.

Beneficial effects of the present disclosure: it is found that TRPM8 receptor agonists and TRPV1-4 and TRPA1 agonists have the effect of masking the bitter taste perception in the oral cavity, and TRPM8 receptor agonists stimulate cold channels, TRPV1-4 and TRPA1 stimulate hot channels, therefore the cool feeling can be used to neutralize the heat feeling and eliminate this pain. The combination of these two drugs completely eliminates the uncomfortable feeling and hot pain and brings a relatively cool and pleasant pleasure.

### BRIEF DESCRIPTION OF THE DRAWINGS

FIG. 1 is a graph showing changes in sodium ion fluorescence concentration of some groups in an immunofluorescence experiment of sodium ion channels;

FIG. 2 is a graph showing changes in sodium ion fluorescence concentration of some groups in an immunofluorescence experiment of sodium ion channels;

FIG. 3 is a graph showing changes in sodium ion fluorescence concentration of some groups in an immunofluorescence experiment of sodium ion channels;

FIG. 4 shows a comparison of the average values of fluorescence pixels of different substances;

FIG. 5 shows a comparison of the average values of fluorescence pixels of different substances; and

FIG. 6 shows a comparison of the average values of fluorescence pixels of different substances.

### DETAILED DESCRIPTION OF EMBODIMENTS

Exemplary embodiments of the present disclosure are described in detail below in conjunction with the accompanying drawings, wherein the same or similar reference numerals represent the same or similar elements. In addition, in the following detailed description, for ease of explanation, many specific details are set forth to provide a comprehensive understanding of the disclosed embodiments. However, it is apparent that one or more embodiments may be implemented without these specific details. In other cases, known structures and devices are embodied in a schematic manner to simplify the drawings.

The inventors have found that, in view of the principle of taste production, sodium ion channel and calcium ion channel blockers may affect taste production. Several members of the TRP family, TRPV1-4, TRPM8 and TRPA1, their antagonists and activators may affect taste production. The relevant product representatives are as follows:

1. Sodium channel blockers: mexiletine, propafenone injection, quinidine sulfate, disopyramide phosphate tablets, moricizine hydrochloride.

2. Calcium channel blockers: dihydropyridine drugs, such as nifedipine, amlodipine, felodipine, etc.; representative drugs of non-dihydropyridines, such as verapamil and diltiazem.

3. Cold channel receptors: cold channel agonists, including TRPM8 agonists and/or TRPA1 agonists. TRPM8 agonists are selected from one or more of menthol, citral, linalool, eugenol and eucalyptol.

Transient receptor potential cation channel subfamily M member 8 (TRPM8), also known as cold and menthol receptor I (CMR1), is a protein expressed in sensory neurons and is activated by low temperature and cooling agents such as menthol and menthol derivatives, mainly including menthyl amide, phenoxyethanol, limonene, 18-cineole, piperine, germacrene, menthyl acetate, WS-3 ((IR, 2S, 5R)-2-isopropyl-5-methyl-cyclohexanecarboxylic acid acetamide), WS-5 ([((IR, 2S, 5R)-2-isopropyl-5-methyl-cyclohexanecarbonyl)-amino]-ethyl acetate), WS-23 (2-isopropyl-2, 3, N-trimethyl-butyramide), menthyl lactate (Frescolat ML), succinic acid menthyl ester, menthoxy propanediol (coolant 10), 2-isopropyl-5-methylcyclohexyl 4-(dimethylamino)-4-oxobutyrate (4-(dimethylamino)-4-oxobutyric acid 2-isopropyl-5-methylcyclohexyl ester).

Clove oil-type substance includes: methyl amyl ketone, clove oil component eugenol (57-85%), caryophyllene (15%), eugenol acetate (18%).

4. TRPA1 agonists are selected from one or more of cinnamaldehyde, mustard oil, allicin, capsaicin ester, carvacrol, eugenol, piperine, gingerol, wintergreen oil, clove oil, caryophyllene and/or caryophyllene oxide, citral or menthol.

TRPV1 agonists are selected from capsaicin, resiniferatoxin, vitamin D, piperine, evodiamine, zanthoxylum, capsaicin esters, 6-shogaol, gingerol, zingerone, zingerone phenol or polygonum dialdehyde.

5. TRPV1 antagonists may include one or more of the following: (−)-bornyl acetate; hydroxycitronellal; aprilonone; methyl N, N-dimethyl anthranilate; 2-ethoxy-3-ethylpyrazine; L-piperidone; isobornyl isobutyrate; 4-acetoxy-2,5-dimethyl-3(2H)-furanone; tripropylamine; dihydrojasmone; 1-methyl-2-pyrrolaldehyde; 3-octyl acetate; 2-methylbutyl isovalerate; piperonyl isobutyrate; phenoxyethyl propionate; vanillin propylene glycol acetate; octenyl cyclopentanone; butyl isobutyrate; guaiac wood oil; tetrahydro-4-methyl-2-(2-methyl-1-propenyl)-2H pyran.

6. TRPA1 antagonists may include one or more of the following: cinnamon bark oil; γ-lauric acid lactone; vanillic acid; γ-methyl decalactone, trans-2,4-nonadienal; 4-allyl-2,6-dimethoxyphenol; o-methoxycinnamaldehyde; 4-methyl-2-phenyl-2-pentenal (mixture of cis and trans); 2-methoxy-4-propylphenol; 2-methoxy-benzoic acid methyl ester; 6-tetradecaprolactone; 1-methyl-2-pyrrolaldehyde; 3,3,5-trimethylcyclohexanol; N-(2-hydroxyethyl) lactamide; 2-(3-phenylpropyl) tetrahydrofuran; anisyl butyrate; methyl 4-phenylbutyrate; 3-heptyldihydro-5-methyl-2(3H)-furanone; 3-acetylthiohexyl acetate; 3-methyl-5-propyl-2-cyclohexene-1-one; isobornyl isobutyrate; bornyl valerate; citronellol acetate; (2S,5S,6S)-6-)hydroxy-dihydroteaspirane; trans-2-hexenal.

The present disclosure is intended to find compounds that can affect taste from these chemicals.

Among the taste evaluation methods, group people taste test was once the preferred method, but this method has strong subjectivity, uncertainty and raw material safety issues. Subsequently, alternative methods such as dissolution, electronic tongue and animal experiments appeared. However, the drugs used in the experiments of this invention are all food grade, are drugs or foods approved by the State Food and Drug Administration, so there is no safety problem, and the results of the experiment should directly guide the application, therefore the taste test of the crowd is selected as the preferred method.

### I. Screening of Substances in the Form of Oral Sprays

Purpose of the experiment: it is to screen out compounds that take effect within 5 seconds, last for 30-120 seconds, and it is expected, by using such kind of compounds, the taste of the drug cannot be felt during the duration (30-120 seconds) when taking the drug.

Principle of the experiment: simulate the process of taking medicine and using the taste masking agents, first spray about 0.5 ml of oral spray on the throat (3 times), and after it takes effect quickly, taste 10 ml of bitter standard solution to screen drugs that can be used in advance and can mask the bitter taste.

### Experimental Steps:

1: Volunteer screening. The research team strictly screened the recruited volunteers and finally selected healthy volunteers as subjects. They signed informed consent forms before the experiment;
2: Standard bitterness sample preparation. The bitterness of the samples was divided into five levels, and each level was assigned a certain bitterness value range;
3: The sensory standard was corrected by the oral taste evaluation method, that is, the sensory standard was corrected by comparing the taste evaluation of the volunteers with the established bitterness value of the reference sample group. Specific steps: when the volunteers are doing the oral tasting test, the reference solutions of various concentrations are placed in the tasting cups, and the volunteers hold one of the solutions in their mouths, time it, and rinse their mouths by using the solution so that the bitter taste perception area at the root and sides of the tongue can fully feel the bitter taste of the drug. They are also informed of the bitterness classification and specific bitterness value of the reference solution, spit it out, rinse their mouths 5 times by water, until there is no bitterness in the mouth, and then measure another concentration of reference solution after 15 minutes, and record the data during this period; due to the strong subjectivity of the oral tasting method, a group of samples with a concentration of 0.025 mmol per liter to be tested is added, which can objectively serve the purpose of examining the reproducibility of the experimental results, that is, by comparing the test results of the two groups of samples with the same concentration before and after, the reproducibility of the test results can be examined;
4: Taste masking experiment. Then dilute all the selected taste masking drugs, prepare the solution, take 10 ml and put it into the spray bottle, spray it into the mouth 3 times, stay for 5 seconds, drink 10 ml of standard bitterness solution, feel and record the bitterness value, keep the standard bitterness solution in the mouth until the bitterness rises, and record the retention time.
5: Quality control. In order to ensure the accuracy of the oral evaluation results, the research team has taken a series of measures to control the evaluation quality, including increasing the sample concentration step by step to reduce the impact of the saturation effect, partially disrupting the sample concentration order to reduce the inertia of the volunteers' consciousness, testing the same sample multiple times to examine the reproducibility, resting the mouth for fifteen to thirty minutes after each tasting, and performing necessary processing on outliers;
6: Processing the experimental data. Since the experimental objects are biological samples and there are individual differences between different subjects, there may be individual outliers in the experimental test data. The Grubbs test method was used to exclude outliers in the data, and then the experimental data were plotted in tables and curves for data analysis.

It is necessary to process the experimental data. Specifically, the Grubbs test method was used to perform a circular test and elimination of outliers on the data, and a bilateral test was used to eliminate outliers in the oral test. Considering the characteristics of biological samples, the detection level was selected as 0.1 and the elimination level was selected as 0.05 in this experiment.

The experimental medicinal material processing method mentioned in the above technical scheme is as follows: Preparation of bitter standard solution, use 10 g of Coptis chinensis powder, add 100 ml of pure water above 90 degrees, stir, let stand for 20 minutes, take the supernatant for use, determine the standard bitterness of 4.0 degrees by oral tasting, dilute 5 times, determine the standard bitterness of 2.0 degrees, dilute 2.0 degrees again 5 times, determine the standard bitterness of 0.5 degrees. Compare with the bitterness scoring table, (level 1 means “no bitterness or almost no bitterness”, the bitterness value is 0.5˜1.5; level 2 means “slightly bitter”, the bitterness value is 1.5˜2.5; level 3 means “bitter but acceptable”, the bitterness value is 2.5˜3.5; level 4 means “very bitter, but still tolerable”, the bitterness value is 3.5˜4.5; level 5 means “unbearable bitterness”, the bitterness value is 4.5˜5.5). Adjust the judgment and cognition of bitterness.

The standard bitterness solution used in this experiment to verify the experimental effect is a 4.0 degree bitterness solution of Coptis chinensis prepared according to the above method. The bitterness perception of volunteers was adjusted through the gradient bitterness solutions, and the following experiment was carried out after determination. 32 people participated in the experiment, 1 group of abnormal data was eliminated, and finally 31 groups of valid data were obtained. The volunteers were healthy, aged 18-30 years old, 7 boys and 25 girls.

The substances to be screened include: linalool, citral, cinnamaldehyde, caryophyllene, eugenol acetate, menthone, succinic menthyl ester, eucalyptus oil, borneol, probenecid, magnesium sulfate, menthamide, cooling agent WS-5, cooling agent WS-23, vanillic acid, hydroxyvanillin, dihydrojasmone, carvacrol, gingerol, phloretin, piperine, allicin, amlodipine, nifedipine, paraviril, dodecalactone, trimethylcyclohexanol, quercetin, eplerenone.

The following table records the data on the changes in bitterness values of various substances to be screened when used as taste-masking sprays during the above experiments.

| Statistics of | | | |
|---|---|---|---|
| bitterness | | | |
| masking effect | Reduction of | Reduction | Duration |
| Substance | bitterness | range | >(s) |
| Linalool | 2.1 | 52.50% | 30 |
| Citral | 2.3 | 57.50% | 30 |
| Cinnamaldehyde | 2.9 | 72.50% | 50 |
| Caryophyllene | 2.3 | 57.50% | 30 |
| Eugenol acetate | 2.4 | 60.00% | 60 |
| Menthone | 2.8 | 70.00% | 30 |
| Menthyl succinate | 1.6 | 40.00% | 10 |
| Eucalyptus oil | 1.1 | 27.50% | 10 |
| Borneolum | 2.1 | 52.50% | 10 |
| Probenecid | 0.5 | 12.50% | 30 |
| Magnesium sulfate | 1.5 | 37.50% | 20 |
| Menthamide | 2.5 | 62.50% | 120 |
| Cooling agent WS-5 | 2.3 | 57.50% | 100 |
| Cooling agent WS-23 | 2 | 50.00% | 30 |
| Vanillic acid | 1.1 | 27.50% | 50 |
| Hydroxyvanillin | 0 | 0.00% | |
| Dihydrojasmone | 0 | 0.00% | |
| Carvacrol | 0 | 0.00% | |
| Gingerol | 2.2 | 55.00% | 20 |
| Phloretin | 0 | 0.00% | |
| Piperine | 2.1 | 52.50% | 30 |
| Allicin | 2.3 | 57.50% | 30 |
| Amlodipine | 0 | 0.00% | |
| Nifedipine | 0 | 0.00% | |
| Paravimide | 0 | 0.00% | |
| Dodecalactone | 0 | 0.00% | |
| Trimethylcyclohexanol | 0 | 0.00% | |
| Quinolide | 0 | 0.00% | |
| Eplerenone | 0 | 0.00% | |
| Blank | 0.2 | 5.00% | 10 |

Description: Case number 31, bitterness reduction value is average value; original bitterness average is 4.0. Each drug is prepared into a 0.2% solution with 60% ethanol solution

Substances (or drugs) with a reduction of less than 5% of the blank solution are considered to be completely ineffective. Substances with a reduction of more than 50% have a taste masking effect that is clinically usable.

### II. Verification of the Broad-Spectrum Taste Masking Effect of Chinese Herbal Medicine Slices

According to the standard of decocting 100 ml of liquid from 10 g of dry Chinese herbal medicine slices (decoction method: use a stainless steel container, and keep for 20 minutes after boiling), from 300 commonly used Chinese herbal medicine slices, combined with the Chinese Pharmacopoeia, more than 20 Chinese herbal medicines with bitter or extremely bitter properties written in the pharmacopoeia were selected, including Bletilla striata, Cortex qin, Stephania tetrandra, Apricot kernel, Belamcanda chinensis, Angelica dahurica, Lotus seed heart, Toosendan fruit, Andrographis paniculata, Sophora flavescens, Coptis chinensis, Gentiana scabra, Forsythia suspensa, Sophora japonica, Phellodendron amurense, Scutellaria baicalensis, Platycodon grandiflorum, Notopterygium wilfordii, Saposhnikovia divaricata, Perilla frutescens, Zanthoxylum bungeana, Osmanthus fragrans, Sophora flavescens, and Chrysanthemum officinale, totally 24 kinds of Chinese herbal medicines.

Finally, through the 5-degree graduation method, 2 species with a bitterness higher than 3.5 (moderate bitterness) and 7 species with a bitterness higher than 5 degrees (highly bitter, extremely bitter, accompanied by nausea) were screened out, a total of 9 species (Angelica pubescens, Lotus Seed Heart, Toosendan, Andrographis paniculata, Sophora flavescens, Coptis chinensis, Gentiana scabra, Forsythia suspensa, Sophora japonica) are selected as bitterness representatives to participate in the bitterness masking experiment. (Note: The bitterness of Coptis chinensis slices can reach 5.2 after 20 minutes of water decoction. The reference standard solution is 90-degree water brewing Coptis chinensis powder, it is automatically cooled to obtain a solution with a bitter taste of 4.0. Because Coptis chinensis has been used as a standard bitter liquid, it is no longer used).

Bitterness reduction value, using 0.1% different taste
masking agents for extremely bitter Chinese medicines

| Chinese | | | | | |
|---|---|---|---|---|---|
| medicine | Original | | | | Menthyl |
| name | bitterness | Citral | Linalool | Cinnamaldehyde | amide |
| Forsythia | 3 | 2.5 | 2 | 1.7 | 1.7 |
| suspensa | | | | | |
| Gentiana | 5 | 3.7 | 1.2 | 4.2 | 1 |
| Sophora | 5 | 1.7 | 0.5 | 2.3 | 1 |
| flavescens | | | | | |
| Lotus seed | 5 | 3.5 | 1.6 | 2.3 | 2.8 |
| heart | | | | | |
| Puhuo | 5 | 3.75 | 2.5 | 4.2 | 2.1 |
| Toulianzi | 5 | 1.5 | 1.1 | 3.5 | 1 |
| Andrographis | 5 | 1.5 | 0.8 | 4 | 0.5 |
| paniculata | | | | | |

Note:
The solvent is 40% ethanol solution

The experimental method is the same as the previous part.

It can be seen from the table above that the taste masking agents listed in the table are all effective for extremely bitter drugs. The best effect is cinnamaldehyde, followed by citral and menthyl amide, and finally linalool.

### III. Immunofluorescence Experiment of Sodium Ion Channels

Observe the changes in sodium ion fluorescence concentration caused by bitter agent stimulation, and judge whether the tested drug has a blocking effect on sodium ion channels based on the results.

### 1.1 Experimental Animals

10 mice (Zhengzhou Huaxing Experimental Animal Co., Ltd.)

### 1.2 Experimental Equipment

Fluorescence microscope BA410 (Motic Industrial Co., Ltd.), TGL-16B centrifuge (Shanghai Anting Scientific Instrument Factory), sterilizer YX-280 (Hefei Huatai Medical Equipment Co., Ltd.), sterile clean bench SW-CJ-1F (Suzhou Huayu Purification Equipment Co., Ltd.), sodium ion fluorescent dye ENG-2 AM (Shanghai McLean Biochemical Technology Co., Ltd.), dispersant Pluronic F-127 (Shanghai McLean), DMSO (Shanghai McLean), DMEM high glucose (Hefei Bomei Biotechnology Co., Ltd.), trypsin (Shanghai McLean), calf serum (Hefei Bomei Biotechnology Co., Ltd.), D-Hanks solution (Hefei Bomei Biotechnology Co., Ltd.).

Menthol, linalool, citral, cinnamaldehyde, eugenol acetate, menthone, menthyl lactate, menthyl succinate, aminobutyric acid, paravirin, vanillic acid, hydroxyvanillic acid, gingerol, piperine, all of the above drugs were provided by Shanghai McLean Biochemical Technology Co., Ltd. Triphenylphosphine oxide TPPO (Hefei Bomei Biotechnology Co., Ltd.); WS-5, WS-23 (Ron reagent); Metronidazole (Shanghai Pulang Biotechnology Co., Ltd.)

Sterilized items: 1 set of surgical instruments, 3 boxes of gun tips (10 ul, 200 ul, 1000 ul), 10 of 2 ml EP tubes and 10 of 1.5 ml EP tubes, 5 culture dishes, 3 of large 50 ml centrifuge tubes, and several glass beakers. Others: physiological saline, sterile small syringe (5 ml), scissors, cell counting plate, culture dish, 95% alcohol, dropper, gun tip (10 ul, 200 ul, 1000 ul), gun, centrifuge tube, gloves, filter paper.

### 1.3 Experimental Steps

### (I) Tongue Removal

The mouse was killed by dislocation, and the tongue was removed (minimizing tongue bleeding), disinfected with alcohol for 2 minutes (adding sodium citrate for anticoagulation), and washed with D-Hanks solution or rinsed with 10 ml of saline solution drawn by a syringe.

### (II) Separation of Cells and Preparation of Cell Suspension

Put the removed tongue in a 2 ml EP tube, add 1-1.5 ml of 0.25% trypsin solution, and incubate in a 37° C. constant temperature incubator for 30 min.

Take out the centrifuge tube from the constant temperature incubator, shake for 2 minutes, blow the undigested tongue several times with the gun tip, take out the tongue, cover it with the EP tube cap, put it in a centrifuge, centrifuge at 3000 rpm for 3 minutes, discard the supernatant, add cell culture medium (containing 10% inactivated serum), blow and disperse the cells, and place it in a 37° C. constant temperature incubator for 30 min to allow the cells to recover.

### (III) Sodium Ion Fluorescence Staining

(1) Take out the prepared ENG-2 AM storage solution and warm it to room temperature for use, keeping it away from light. The concentration of the pre-prepared stock solution is 2 mM and it is stored at −18° C. for future use.

(2) Dilute the ENG-2 AM+DMSO stock solution to a 1-10M working solution using serum-free culture medium.

Ratio process: 5 ul of 2 mM stock solution+10 ul of Pluronic F-127 stock solution+980 ul of serum-free culture medium, the final concentration is about 1 μM.

(3) Load the probe in a serum-free culture medium containing an appropriate concentration of AM probe.

Take out the cells from the incubator, centrifuge, and discard the culture medium. Wash the cells once with D-Hanks solution, centrifuge, and discard the culture medium. Add 1 ml of culture medium again, then add 300 ul of sodium ion probe dilution solution, blow and disperse the cells, and incubate the cells at room temperature or 37° C. for 1 hour.

At the end of incubation, centrifuge at 100 rpm for 2 minutes, discard the supernatant, add 500 ul of cell culture medium without AM probe, blow and disperse the cells, and incubate for 20-60 min to ensure that the cells completely deesterify the AM group.

(4) Take 10 ul of incubation solution and drop it into the center of the cell counting plate, cover it with a slide, and observe it under a fluorescence microscope.

(5) Use a low-power microscope to find the cells, open the fluorescence path, detect the fluorescence signal with a fluorescence microscope, change to a high-power microscope, find the appropriate observation cells, observe and take pictures to record the initial signal.

### (IV) Detect Drug Effects (the Bitter Drug Tested is a 2% Metronidazole Solution)

(1) Blank control: Add 5 ul of drug solvent (53% alcohol solution) to one side of the cover glass on the cell counting plate, use absorbent paper to absorb water on the opposite side, let the solution soak the cells, and then use a fluorescence microscope to observe and take pictures 3-5 times, one every 1-2 minutes; then add 5 ul of standard bitter drug to one side of the cover glass on the cell counting plate, use absorbent paper to absorb water on the opposite side, let the solution soak the cells, and then use a fluorescence microscope to observe and take pictures 3-5 times, one every 1-2 minutes.

(2) Experimental drugs: Add 5 ul of the tested drug to one side of the cover glass on the cell counting plate, use absorbent paper to absorb water on the opposite side, let the solution soak the cells, and then use a fluorescence microscope to observe and take 3-5 photos, one every 1-2 minutes; then add 5 ul of the standard bitter drug to one side of the cover glass on the cell counting plate, use absorbent paper to absorb water on the opposite side, let the solution soak the cells, and then use a fluorescence microscope to observe and take 3-5 photos, one every 1-2 minutes.

The tested drugs include: menthol, linalool, citral, cinnamaldehyde, eugenol acetate, menthone, menthyl lactate, menthyl succinate, aminobutyric acid, paravirin, vanillic acid, hydroxyvanillic acid, gingerol, piperine.

(3) Blocker control: Authoritative research believes that TRPM5 blocker, triphenylphosphine oxide (TPPO), is a reversible and selective TRPM5 channel blocker; finally, triphenylphosphine oxide (TPPO) is used as the test drug and the above steps are repeated.

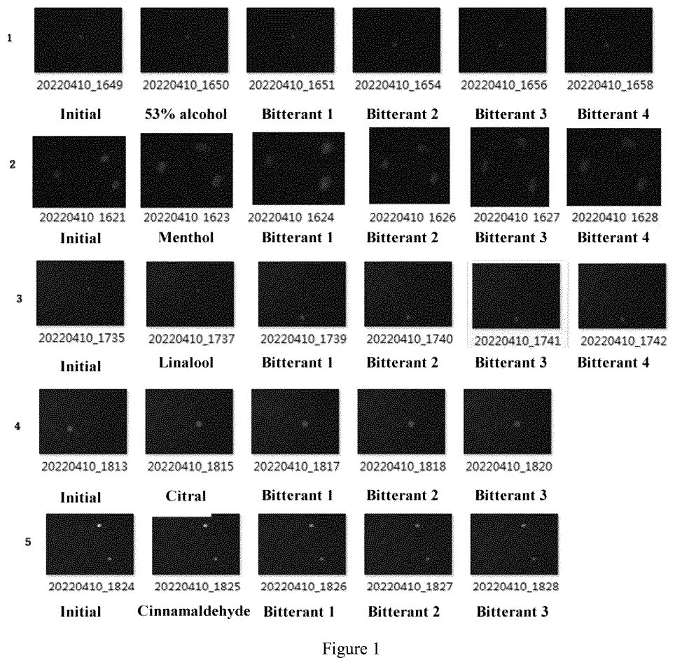

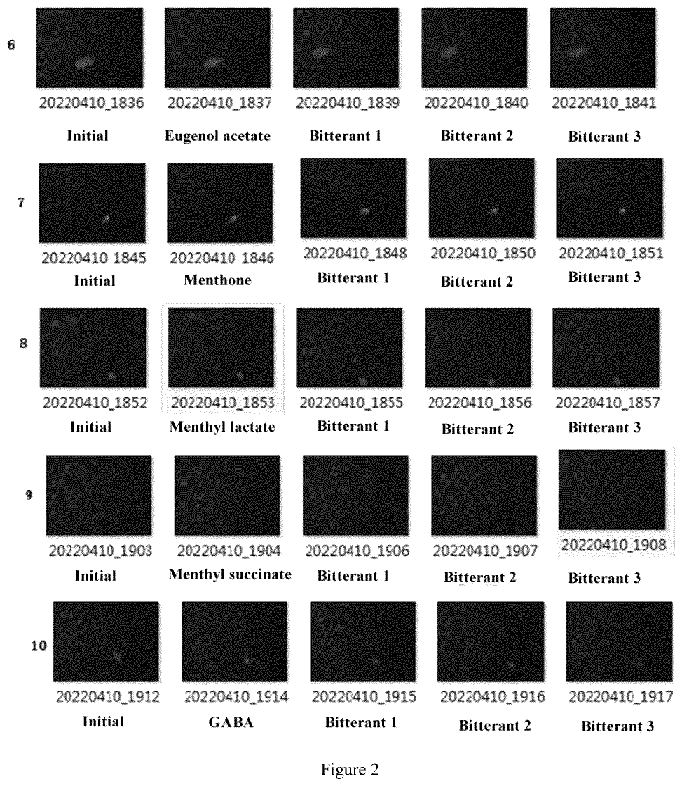

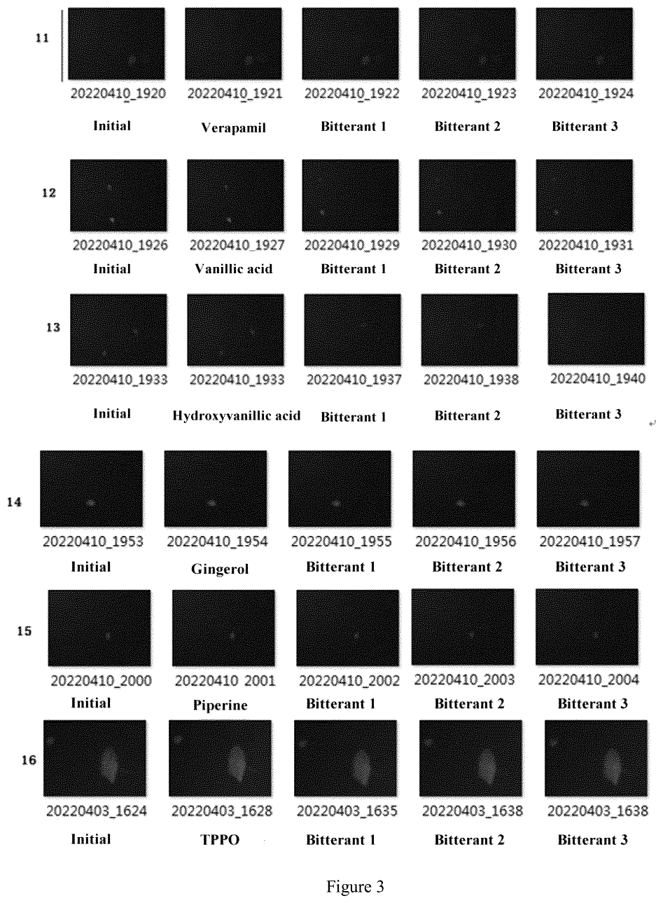

(4) Observation and analysis: Through the statistical observation of the changes in sodium ion fluorescence in the above process, including the time point of action, duration, intensity and other factors, the added screening compounds are compared to observe the changes in sodium ion fluorescence concentration caused by the stimulation of standard bitter agents, and the results are used to judge whether the test drug has a blocking effect on the sodium ion channel. The results are shown in FIGS. 1-3.

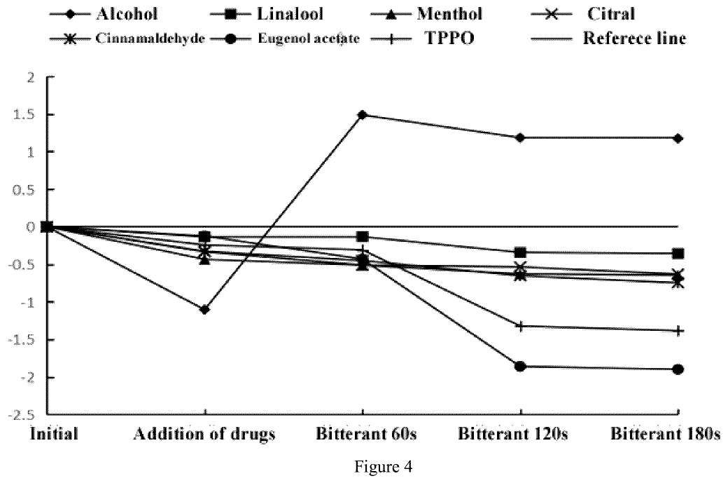

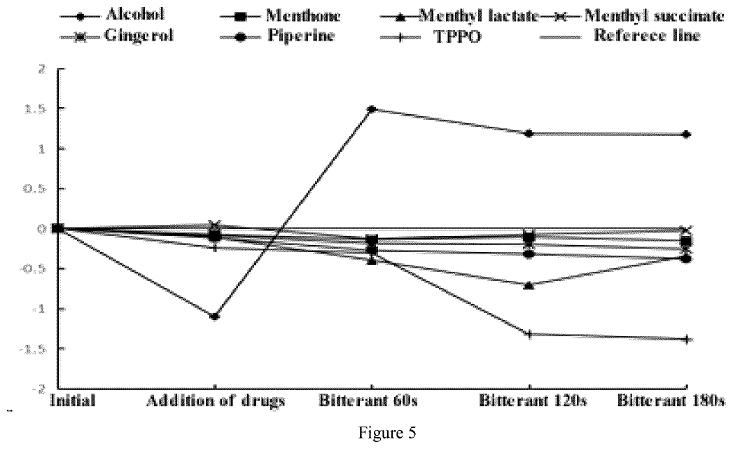

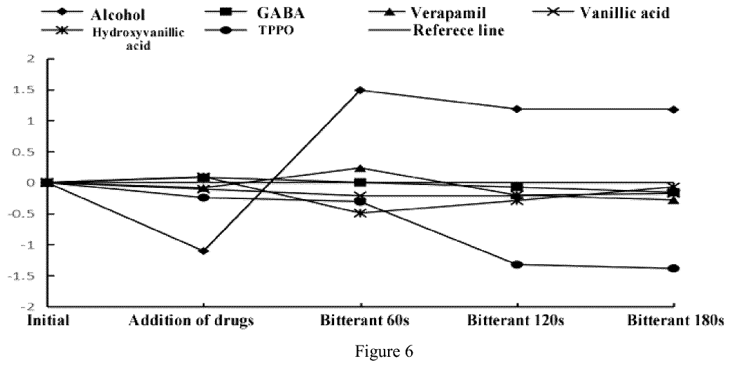

(5) Data analysis: Use Image J-Fiji software to analyze the images, compare the average values of the fluorescence pixels, remove the base number and compare them in the table, draw FIGS. 4-6, and use SPSS18.0 to perform chi-square tests to compare the differences between the data groups.

Comparative analysis of the average values of fluorescence brightness data

| | Ethanol as | | | | | Eugenol |
|---|---|---|---|---|---|---|
| | reference | Linalool | Citral | Cinnamaldehyde | Menthol | acetate |
| Initial | 20.682 | 27.783 | 30.278 | 31.345 | 22.553 | 28.228 |
| Addition | 19.578 | 27.648 | 29.943 | 31.017 | 22.118 | 28.103 |
| of drugs | | | | | | |
| Bitterant | 22.169 | 27.649 | 29.766 | 30.893 | 22.04 | 27.797 |
| 60 s | | | | | | |
| Bitterant | 21.866 | 27.44 | 29.742 | 30.691 | 21.924 | 26.368 |
| 120 s | | | | | | |
| Bitterant | 21.856 | 27.426 | 29.647 | 30.597 | 21.907 | 26.329 |
| 180 s | | | | | | |
| Bitterant | 21.845 | 26.46 | | | 20.562 | |
| 240 s | | | | | | |

| | | Menthyl | Menthyl | | |
|---|---|---|---|---|---|
| | Menthone | lactate | succinate | GABA | Verapamil |
| Initial | 25.556 | 28.538 | 28.879 | 27.285 | 27.855 |
| Addition | 25.48 | 28.425 | 28.92 | 27.367 | 27.77 |
| of drugs | | | | | |
| Bitterant | 25.415 | 28.141 | 28.747 | 27.287 | 28.089 |
| 60 s | | | | | |
| Bitterant | 25.447 | 27.831 | 28.8 | 27.21 | 27.651 |
| 120 s | | | | | |
| Bitterant | 25.4 | 28.191 | 28.851 | 27.127 | 27.574 |
| 180 s | | | | | |
| | Vanillic | Hydroxyvanillic | | | Triphenylphosphine |
| | acid | acid | Gingerol | Piperine | oxide |
| Initial | 26.902 | 27.989 | 25.337 | 26.358 | 88.898 |
| Addition | 26.795 | 28.082 | 25.244 | 26.237 | 88.789 |
| of drugs | | | | | |
| Bitterant | 26.689 | 27.496 | 25.15 | 26.082 | 88.608 |
| 60 s | | | | | |
| Bitterant | 26.699 | 27.697 | 25.137 | 26.037 | 88.598 |
| 120 s | | | | | |
| Bitterant | 26.725 | 27.919 | 25.08 | 25.978 | 88.583 |
| 180 s | | | | | |

Comparative analysis of the average values of fluorescence brightness data

| | Ethanol as | | | | | Eugenol |
|---|---|---|---|---|---|---|
| | reference | Linalool | Menthol | Citral | Cinnamaldehyde | acetate |
| Initial | 0 | 0 | 0 | 0 | 0 | 0 |
| Addition | −1.104 | −0.135 | −0.435 | −0.335 | −0.328 | −0.125 |
| of drugs | | | | | | |
| Bitterant | 1.487 | −0.134 | −0.513 | −0.512 | −0.452 | −0.431 |
| 60 s | | | | | | |
| Bitterant | 1.184 | −0.343 | −0.629 | −0.536 | −0.654 | −1.86 |
| 120 s | | | | | | |
| Bitterant | 1.174 | −0.357 | −0.646 | −0.631 | −0.748 | −1.899 |
| 180 s | | | | | | |
| Bitterant | 1.163 | −1.323 | −1.991 | | | |
| 240 s | | | | | | |

| | | Menthyl | Menthyl | | |
|---|---|---|---|---|---|
| | Menthone | lactate | Succinate | Gingerol | Piperine |
| Initial | 0 | 0 | 0 | 0 | 0 |
| Addition | −0.076 | −0.113 | 0.041 | −0.093 | −0.121 |
| of drugs | | | | | |
| Bitterant | −0.141 | −0.397 | −0.132 | −0.187 | −0.276 |
| 60 s | | | | | |
| Bitterant | −0.109 | −0.707 | −0.079 | −0.2 | −0.321 |
| 120 s | | | | | |
| Bitterant | −0.156 | −0.347 | −0.028 | −0.257 | −0.38 |
| 180 s | | | | | |
| | | | Vanillic | Hydroxyvanillic | Triphenylphosphine |
| | GABA | Verapamil | acid | acid | oxide |
| Initial | 0 | 0 | 0 | 0 | 0 |
| Addition | 0.082 | −0.085 | −0.107 | 0.093 | −0.243 |
| of drugs | | | | | |
| Bitterant | 0.002 | 0.234 | −0.213 | −0.493 | −0.308 |
| 60 s | | | | | |
| Bitterant | −0.075 | −0.204 | −0.203 | −0.292 | −1.322 |
| 120 s | | | | | |
| Bitterant | −0.158 | −0.281 | −0.177 | −0.07 | −1.384 |
| 180 s | | | | | |

Result analysis: Group 1 is blank control group (for reference), after adding bitter agent, sodium ion channel is activated, intracellular fluorescence is enhanced, and then the fluorescence decays naturally over time; Group 16, after adding triphenylphosphine oxide, sodium ion channel is blocked, intracellular fluorescence does not increase, but decays over time. Comparing with other groups, compounds similar to triphenylphosphine oxide TPPO are found, they are compounds that can block the sodium ion channel of Trpm5 bitter receptor. When bitter drug is added to groups 2-9 and 14-15 at 0S, the fluorescence does not increase, and then they all maintain a decay trend; that is, menthol, linalool, citral, cinnamaldehyde, eugenol acetate, menthone, menthyl lactate, menthyl succinate, gingerol, and piperine all have effects similar to triphenylphosphine oxide TPPO, they are compounds that can block the sodium ion channel of Trpm5 bitter receptor. When bitter drug is added to groups 10, 11, and 13 at 0S, the fluorescence increases, exceeds the baseline, and then maintains a decay trend; while when bitter drug is added to group 12 for 0S, the fluorescence does not increase, but when 120S passes, it increases against the trend and exceeds the baseline. Therefore, it is believed that aminobutyric acid, paravir, vanillic acid, and hydroxyvanillic acid have poor effects and do not have bitterness masking effect.

Using SPSS18.0 to perform a chi-square test to compare the differences in data groups, it was found that groups 2-9 and 14-15 had no difference with the group 16 of triphenylphosphine oxide (p>0.05), but were significantly different from the blank group (p<0.01); while groups 10-13 were the opposite, they are different from the group 16 of triphenylphosphine oxide (p<0.05), but with no difference from the blank group (p>0.05). These verified the above results.

Cold channel agonists include TRPM8 agonists and/or TRPA1 agonists, and typical representatives include menthol, linalool, citral, cinnamaldehyde, eugenol acetate, menthone, menthyl lactate, menthyl succinate, gingerol, and piperine, which are the compounds verified by our experiments. Whether it is a tasting experiment or a fluorescence experiment, because of the consistency of the action principle, we believe that this class of compounds can act on TRPM5 and block the production of bitterness.

The representative products of TRPV1 antagonists and TRPA1 antagonists, aminobutyric acid, paravir, vanillic acid, and hydroxyvanillic acid, do not have the effect of masking bitterness and acting on TRPM5. This has been verified in both tasting experiments and fluorescence experiments. Because of the consistency of the action principle, we believe that this class of compounds cannot act on TRPM5 and cannot block the production of bitterness.

Sodium channel blockers and calcium channel blockers are used to treat heart and vascular diseases. The principle is also related to sodium ion and calcium ion channels, but it is experimentally verified that they cannot mask the bitterness.

Through the above experiments, it is speculated that:

### (1) TRPM8 Agonists are Effective

Transient receptor potential cation channel subfamily M member 8 (TRPM8), also known as cold and menthol receptor I (CMR1), is a protein expressed in sensory neurons and is activated by low temperature and cooling agents such as menthol and menthol analogs, including menthol, citral, linalool, eugenol, eucalyptol, menthol, eugenol, limonene, 18-cineole, piperine, germacrene, menthyl acetate, synthetic WS-3 ((IR, 2S, 5R)-2-isopropyl-5-methyl-cyclohexanecarboxylic acid acetamide), WS-5 ([((IR, 2S, 5R)-2-isopropyl-5-methyl-cyclohexanecarbonyl)-amino]-ethyl acetate), WS-23 (2-isopropyl-2, 3, N-trimethyl-butyramide), menthyl lactate (Frescolat ML), menthoxypropanediol (coolant 10), 2-isopropyl-5-methylcyclohexyl 4-(dimethylamino)-4-oxobutyrate (4-(dimethylamino)-4-oxobutyric acid 2-isopropyl-5-methylcyclohexyl ester), methyl amyl ketone, caryophyllene, eugenol acetate.

### (2) TRPA1 Agonists are Effective

Representative drugs include: one or more of cinnamaldehyde, mustard oil, allicin, capsaicin ester, carvacrol, eugenol, piperine, gingerol, wintergreen oil, clove oil, caryophyllene and/or caryophyllene oxide, citral and menthol.

### (3) TRPV1-4 Agonists are Effective

Representative drugs include:

TRPV1 agonists are selected from capsaicin, resin toxin, vitamin D, piperine, evodiamine, prickly ash, capsaicin ester, 6-shogaol, gingerol, gingerone, gingerol or water polygonal dialdehyde.

### (4) TRPV1 Antagonists and TRPA1 Antagonists May be Ineffective

TRPV1 antagonists include one or more of the following: (−)-bornyl acetate; hydroxycitronellal; aprilonone; methyl N, N-dimethyl anthranilate; 2-ethoxy-3-ethylpyrazine; L-piperidone; isobornyl isobutyrate; 4-acetoxy-2,5-dimethyl-3(2H)-furanone; tripropylamine; dihydrojasmone; 1-methyl-2-pyrrolaldehyde; 3-octyl acetate; 2-methylbutyl isovalerate; piperonyl isobutyrate; phenoxyethyl propionate; vanillin propylene glycol acetate; octenyl cyclopentanone; butyl isobutyrate; guaiac wood oil; tetrahydro-4-methyl-2-(2-methyl-1-propenyl)-2H-pyran.

TRPA1 antagonists include one or more of the following: γ-lauric acid lactone; vanillic acid; γ-methyl decanolide; trans-2,4-nonadienal; 4-allyl-2,6-dimethoxyphenol; o-methoxycinnamaldehyde; 4-methyl-2-phenyl-2-pentenal (mixture of cis and trans); 2-methoxy-4-propylphenol; 2-methoxy-benzoic acid methyl ester; 6-tetradecaprolactone; 1-methyl-2-pyrrolaldehyde; 3,3,5-trimethyl Cyclohexanol; N-(2-hydroxyethyl) lactamide; 2-(3-phenylpropyl) tetrahydrofuran; anisyl butyrate; methyl 4-phenylbutyrate; 3-heptyldihydro-5-methyl-2(3H)-furanone; 3-acetylthiohexyl acetate; 3-methyl-5-propyl-2-cyclohexen-1-one; isobornyl isobutyrate; bornyl valerate; citronellol acetate; (2S,5S,6S)-6-)hydroxy-dihydroteaspirane; trans-2-hexenal.

### IV. Use Concentration and Combination to Get the Best Effect

Statistics of different concentrations of drugs to reduce bitterness

| | Initial bitterness 4.0, bitterness value after drug | | | | |
|---|---|---|---|---|---|
| Substance | 0.001% | 0.005% | 0.02% | 0.10% | 0.50% |
| Linalool | 3.5 | 2.5 | 2.1 | 1.9 | 1.9 |
| Citral | 3.7 | 2.8 | 2.1 | 1.7 | 1.9 |
| Cinnamaldehyde | 3.5 | 2.6 | 1.5 | 1.1 | 1.2 |
| Caryophyllene | 3.8 | 2.6 | 1.9 | 1.7 | 1.8 |
| Eugenol acetate | 3.8 | 3.2 | 2.3 | 1.6 | 1.4 |
| Menthone | 2.5 | 2.1 | 1.4 | 1.2 | 1.1 |
| Menthyl succinate | 3.2 | 2.8 | 2.6 | 2.4 | 2.1 |
| Eucalyptus oil | 4 | 3.5 | 3.2 | 2.9 | 2.9 |
| Borneolum | 4 | 3.4 | 2.6 | 1.9 | 2.2 |
| Magnesium sulfate | 3.5 | 3.2 | 2.8 | 2.5 | 2.3 |
| Menthamide | 2.4 | 1.8 | 1.5 | 1.5 | 1.6 |
| Cooling agent WS-5 | 2.6 | 1.8 | 1.7 | 1.7 | 1.8 |
| Cooling agent WS-23 | 2.5 | 2.4 | 2.1 | 2 | 2.1 |
| Gingerol | 4 | 2.4 | 2.1 | 1.8 | 1.8 |
| Piperine | 3.9 | 2.5 | 2.3 | 1.9 | 1.9 |
| Garlicin | 3.9 | 3.2 | 2.3 | 1.7 | 1.5 |

Description: Case number 16, bitterness reduction value is average value; original bitterness average is 4.0. Each drug is prepared with 60% ethanol solution.

The concentration used is between 0.00100 and 0.5%. Generally, the effect is better as the concentration increases. However, the effect is not obvious when the concentration increases to about 0.1%, and the irritation is getting greater and greater. Therefore, whether it is economical or practical, it is more appropriate to choose a lower concentration with better effect.

The application of TRPM8 agonist produces a cold sensation, but the provision of TRPV1-4 and TRPA1 agonists can significantly inhibit it, indicating that there is a restrictive relationship between the “cold and hot” channels, and they can both act on the M5 receptor to cause bitterness inhibition. TRPV 1-4 and TRPA1 agonists can act on pain receptors to produce a hot and spicy pain sensation, and when the cool sensation is used to neutralize the heat sensation, this painful sensation is weakened, and the cool sensation brings a kind of pleasure. Therefore, the preferred is combination of these two, avoiding the hot and spicy pain sensation while masking the bitterness, leaving only a relatively cool pleasure.

Our preferred combination of TRPM8 receptor activator and TRPV1-4 and TRPA1 activator can reduce irritation and injury.

You can also use one alone. Although it is irritating, the stimulation can bring people a sense of pleasure afterwards.

### V. Effects of Specific Formulas

Substance A (TRPM8 agonist) and substance B (TRPA1 agonist), A+B+sweetener+flavor; or A or B alone+sweetener+flavor.

A+B+sweetener+flavor is preferred.

For example: 0.05% menthamide, 0.2% cinnamaldehyde, 0.1% sucralose, 0.5% cyclamate, and appropriate amount of flavor.

| Chinese medicine | Original | Reduced |
|---|---|---|
| name | bitterness | bitterness |
| Forsythia | 3 | 0 |
| suspensa | | |
| Gentiana | 5 | 0.5 |
| Sophora | 5 | 0.8 |
| flavescens | | |
| Lotus seed heart | 5 | 0.9 |
| Angelica dahurica | 5 | 0 |
| Toosendan | 5 | 0.5 |
| Andrographis | 5 | 1 |
| paniculata | | |

For these bitter drugs, basically the bitterness is no longer felt. There is still a slight sense of bitterness above 0.5 degrees.

Beneficial effects of the present disclosure: it is found that TRPM8 receptor agonists and TRPV1-4 and TRPA1 agonists have the effect of masking the bitter taste perception in the oral cavity, and TRPM8 receptor agonists stimulate cold channels, TRPV1-4 and TRPA1 stimulate hot channels, therefore the cool feeling can be used to neutralize the heat feeling and eliminate this pain. The combination of these two drugs completely eliminates the uncomfortable feeling and hot pain and brings a relatively cool and pleasant pleasure.

These drugs can be used in a variety of preparations, providing an additional option for drug palatability. The suitable concentration range is 0.001%-0.5%. The preparations that mask the bitterness can also include solvents, sweeteners, preservatives and spices. In particular, sweeteners and spices can be used to neutralize the unpleasant taste of Class A substance and Class B substance themselves. In addition, the taste masking agent can be used in drugs, food or oral care products.

Herein, when the taste masking agent is used for drugs, the drugs include any one of anti-tumor drugs, drugs for treating hyperlipidemia, drugs for treating hyperglycemia, analgesics, antibiotics, antipyretics and traditional Chinese medicines, and the dosage forms of the drugs include any one of tablet, decoction, wine preparation, granule, liquid oral preparation and aerosol. When the taste masking agent is used for food, the food includes any one of ice cream, custard, emulsion, beverages and cake. When the taste masking agent is used for oral care products, the oral care products include any one of toothpaste, mouthwash, chewing gum, breath freshener and mouthwash agent.

The tablets may include any one of multilayer tablets, chewable tablets, soluble tablets, effervescent tablets, sustained-release tablets, controlled-release tablets, dispersible tablets, lozenges, orodisintegrating tablets, sublingual tablets and oral patches.

General term definition: In this application, “TRPM8” has multiple synonyms: TRPP8, LTRPC6.CMRI, MGC2849, transient receptor potential cation channel subfamily M member 8. All functional modifications of the receptor are also included, for example splice variants, subtypes, such as TRPM8CRA-a.TRPM8CRA-b.

In this application, “TRP” is the same as “trp” or “Trp”, which is the abbreviation of Transient receptor potential Cation channel, and there is no difference in the meaning of uppercase and lowercase letters.

In the content of the present disclosure, Chinese herbal medicines are selected according to the Chinese Pharmacopoeia-2020 edition.

Forsythia suspensa: This product is the dried fruit of Forsythia suspensa (Thunb.) Vahl, a plant of the Oleaceae family. In autumn, the fruit is harvested when it is just ripe and still green, impurities are removed, steamed, and dried in the sun, then it is commonly known as “green forsythia”; the fruit is harvested when it is ripe, dried in the sun, and impurities are removed, then it is commonly known as “old forsythia”. This product, calculated and weighted on a dry basis, contains no less than 0.15% forsythin (C₂₇H₃₄O₁₁).

Gentiana: This product is the dried roots and rhizomes of Gentiana manshurica Kitag., Gentiana scabra Bge., Gentiana triflora Pall. or Gentiana rigescens Franch. of the Gentianaceae family. The first three are commonly known as “gentian” and the latter is commonly known as “rigid gentian”. It is picked up in spring and autumn, washed and dried. Calculated and weighted on a dry basis, the content of gentiopicroside (C₁₆H₂₀O₉) in the gentiana must not be less than 3.0%; the content of gentiopicroside (C₁₆H₂₀O₉) in the rigid gentian must not be less than 1.5%.

Toosendan fruit: This product is the dried mature fruit of Melia toosendan Sieb.et Zucc. of the Meliaceae family. It is harvested in winter when the fruit is ripe, and the impurities are removed and then it is dried. This product, calculated and weighted on a dry basis, should contain 0.060% to 0.20% toosendanin (C₃₀H₃₈O₁₁).

Lotus seed heart: This product is the dried young leaves and radicles of the mature seeds of the lotus plant Nelumbo nucifera Gaertn. of the Nelumbo family. Take it out and dry it in the sun. This product, calculated and weighted on a dry basis, should contain no less than 0.70% neenergine (C₃₈H₄₅N₂O₆).

Andrographis paniculata: This product is the dried aerial part of the Andrographis paniculata (Burm.f) Nees of the Acanthaceae family. It is harvested in early autumn when the stems and leaves are lush and dried in the sun. This product, calculated and weighted on a dry basis, contains andrographolide (C₂₀NH₃₀O₅), neoandrographolide (C₂₀H₃₀O₈), 14-deoxyandrographolide (C₂₀H₃₀O₄) and dehydroandrographolide (C₂₀H₂₈O₄) in a total amount of not less than 1.5%.

Sophora tonkinensis: This product is the dried root and rhizome of the leguminous plant Sophora tonkinensis Gagnep. It is harvested in autumn, impurities are removed, and then washed and dried. [Content determination] The total amount of matrine (C₁₅H₂₄N₂₀) and oxymatrine (C₁₅H₂₄N₂O₂) contained in the same medicinal material shall not be less than 0.60%.

Angelica pubescens: This product is the dried root of Angelica pubescens Maxim.f. biserrata Shan et Yuan, a plant of the Umbelliferae family. Dig up in early spring when the seedlings just sprout or in late autumn when the stems and leaves wither, remove the fibrous roots and mud, dry until half dry, pile for 2 to 3 days, and dry again after softening. This product, calculated and weighted on a dry basis, contains no less than 0.50% osthole (C₁₅H₁₆O₃) and no less than 0.080% dihydro-eugenol angelic acid ester (C₁₉H₂₀O₅).

The following table shows the Chinese names, aliases, and molecular formulas of some chemical drugs for easy comparison.

| | | Introduction to triphenylphosphine oxide | |
|---|---|---|---|
| Introduction to cinnamaldehyde | | (TPPO) | |
| Chinese | | Chinese | Triphenylphosphine |
| name: | Cinnamaldehyde | name: | oxide |
| Chinese | Natural | Chinese | Triphenylphosphine |
| alias: | cinnamaldehyde; β | alias: | oxide |
| | phenylpropenal; | Molecular | C₁₈H₁₅OP |
| | Cinnamaldehyde; | formula: | |
| | Trans-cinnamaldehyde; | | |
| | Cinnamaldehyde; 3- | | |
| | phenyl-2-propenal | | |
| Molecular | C₉H₈O | | |
| formula: | | | |
| Introduction to menthol | | Introduction to allicin | |
| Chinese | | Chinese | |
| name: | (+/−)-menthol | name: | Diallyl trisulfide |
| Chinese | 2-isopropyl-5- | Chinese | Diallyl trisulfide; |
| alias: | methylcyclohexanol; | alias: | Allicin |
| | Menthol; Racemic | Molecular | C₆H₁₀S₃ |
| | menthol; DL-menthol; L- | formula: | |
| | (−)-menthol; Menthol | | |
| Molecular | C₂₀H₄₀O₂ | | |
| formula: | | | |
| Introduction to linalool | | Introduction to gingerol | |
| Chinese | | Chinese | |
| name: | linalool | name: | 6-gingerol |
| Chinese | linalool | Chinese | (E)-1-(4-hydroxy-3- |
| alias: | | alias: | methoxyphenyl)dec-4-ene-3-one |
| Molecular | C₁₀H₁₈O | Molecular | C₁₇H₂₄O₃ |
| formula: | | formula: | |
| Introduction to citral | | Introduction to caryophyllene | |
| Chinese | | Chinese | |
| name: | citral | name: | caryophyllene |
| Chinese | 3,7-dimethyl-2,6- | Chinese | trans-caryophyllene |
| alias: | octadiene; natural citral | alias: | |
| Molecular | C₂₀H₃₂O₂ | Molecular | C₁₄H₂₂ |
| formula: | | formula: | |
| Introduction to menthone | | Introduction to eugenol acetate | |
| Chinese | 5-methyl-2- | Chinese | |
| name: | isopropylcyclohexanone | name: | Eugenol acetate |
| Chinese | Menthone; 1-methyl-4- | Chinese | 4-allyl-2-methoxyphenyl |
| alias: | isopropylcyclohexanone- | alias: | |
| | [3]; trans-5-methyl-2-(1- | Molecular | C₁₂H₁₄O₃ |
| | methylethyl)cyclohexanone; | formula: | |
| | Menthone (mixed with | | |
| | isomers); [Grass (above) + | | |
| | Menthyl] ketone; 5- | | |
| | methyl-2-(1- | | |
| | methylethyl)cyclo-hexanone | | |
| Molecular | C₁₀H₁₈O | | |
| formula: | | | |
| Introduction to menthyl lactate | | Introduction to borneol | |
| Chinese | | Chinese | |
| name: | Menthyl lactate | name: | Camphor |
| Chinese | 2-hydroxypropionic acid-5- | Chinese | Borneol; 2-borneol |
| alias: | methyl-2-(1- | alias: | |
| | methylethyl)cyclohexyl | Molecular | C₁₀H₁₆O |
| | ester; (−)-menthol lactate; | formula: | |
| | Menthyl lactate; L- | | |
| | menthyl lactate | | |
| Molecular | C₁₃H₂₄O₃ | | |
| formula: | | | |
| Introduction to piperine | | Introduction to eucalyptus oil | |
| Chinese | | | lemon eucalyptus |
| name: | piperine | Chinese name: | oil |
| Chinese | (E,E)-1-[5-(1,3- | Chinese alias: | eucalyptus oil; |
| alias: | benzodioxol-5-yl)-1-oxo-2,4- | | eucalyptus oil |
| | pentadieny1]- | Molecular | C₁₀H₁₈O |
| | piperidine | formula: | |
| Molecular | C₁₇H₁₉NO₃ | | |
| formula: | | | |
| | | Introduction to | |
| Introduction to vanillic acid | | menthyl acetate ester | |
| Chinese | | Chinese | |
| name: | vanillic acid | name: | menthyl acetate |
| Chinese | vanillic acid; 3-methoxy-4- | Chinese | 5-methyl-2-(1- |
| alias: | hydroxybenzoic acid; 4- | alias: | methylethyl) |
| | hydroxy-3- | | cyclohexanol |
| | methoxybenzoic acid | | acetate; (1R)-(−)- |
| Molecular | C₈H₇O₄ | | menthyl acetate; L- |
| formula: | | | menthyl acetate; |
| | | | Acetic acid 5- |
| | | | methyl-2-(1- |
| | | | methylethyl) |
| | | | cyclohexyl ester; 5- |
| | | | methyl-2-(1- |
| | | | methylethyl) |
| | | | cyclohexane acetate |
| | | Molecular | C₁₂H₂₂O₂ |
| | | formula: | |
| Introduction to aminobutyric acid | | Introduction to menthyl amide | |
| Chinese | | Chinese | N-ethyl-L-menthyl |
| name: | 4-aminobutyric acid | name: | carboxamide |
| Chinese | gamma-aminobutyric acid; | Chinese | N-ethyl-5-methyl-2- |
| alias: | γ-aminobutyric acid; | alias: | (1-methylethyl) |
| | aminobutyric acid; | | cyclohexanecarboxa |
| | 4(GAMMA)-aminobutyric | | mide; Menthyl |
| | acid (aminobutyric acid); | | amide; Cooling |
| | aminobutyric acid; | | agent ws-3; N-ethyl- |
| | gamma-aminobutyric acid; | | p-menthyl-3- |
| | butyric acid; aminobutyric | | carboxamide |
| | acid; GABA | Molecular | C₁₃H₂₅NO |
| Molecular | C₄H₉NO₂ | formula: | |
| formula: | | | |
| | | Introduction to | |
| Introduction to verapamil | | Cooling Agent WS-23 | |
| Chinese | | | N,2,3-trimethyl-2- |
| name: | Verapamil | Chinese | isopropylbutyramide |
| Chinese | Verapamil; Isopamine; 5- | name: | N,2,3-trimethyl-2- |
| alias: | ((3,4- | Chinese | (1-methylethyl)- |
| | dimethoxyphenylethyl)met | alias: | butyramide; |
| | hylamino)-2-(3,4- | | Cooling Agent WS- |
| | dimethoxyphenyl)-2- | | 23 |
| | isopropylvaleronitrile | Molecular | C₁₀H₂₁NO |
| Molecular | C₂₇H₃₇N₂O₄ | formula: | |
| formula: | | | |

“Masking bitterness” refers to the use of a drug (whether used before or at the same time) so that the user does not feel bitterness or the value of bitterness is reduced. “Taste masking” refers to the use of a drug (whether used before or at the same time) so that the user does not feel bitterness or the value of bitterness is reduced.

“Agonists” or “activators” are also called stimulants, which are molecules such as drugs, enzyme agonists and hormones that can enhance the activity of another molecule and promote a certain reaction.

“Antagonists” do not cause biological effects themselves after binding to receptors, but block the effects mediated by the receptor agonists.

The compounds can exist in chemically pure or enriched form, in the form of a single stereoisomer or a mixture of stereoisomers. In addition, the compounds can be uncharged or in the form of salts, such as acid addition salts. Functional groups can be optionally replaced by equivalent chemical groups; fluorine atoms can be replaced by other halogen atoms, such as Cl, Br or I; oxygen atoms (such as ether groups) can be replaced by corresponding sulfur groups, and vice versa; ketone groups can be replaced by corresponding sulfinyl groups. The compounds specifically described above are chemical substances known per se and can be purchased commercially or obtained by conventional organic synthesis methods. If the modified forms or derivatives also exhibit the desired biological activity, they are also functional analogs or functional equivalent compounds.

The concentrations mentioned in the present disclosure, unless otherwise specified, are all volume concentrations.

Although the embodiments of the present disclosure have been shown and described, it is understood by those skilled in the art that these embodiments can be changed without departing from the principles and spirit of the present disclosure. The scope of the present disclosure is defined by the attached claims and their equivalents.

What is claimed is:

1. A method comprising administration of an effective amount of agonist as a substance for masking bitterness, wherein,
the agonist is TRPM8 agonist, the TRPM8 agonist comprises one or more of the following substances: menthol derivatives, citral, linalool, eugenol or eucalyptol;
wherein the menthol derivative comprises one or more of the following substances: menthyl amide, phenoxyethanol, limonene, 18-cineole, piperine, geranyl, menthyl acetate, WS-3 ((IR, 2S, 5R)-2-isopropyl-5-methyl-cyclohexanecarboxylic acid acetamide), WS-5 ([((IR, 2S, 5R)-2-isopropyl-5-methyl-cyclohexanecarbonyl)-amino]-ethyl acetate), WS-23 (2-isopropyl-2, 3, N-trimethyl-butyramide), menthyl lactate, menthyl succinate, menthoxypropanediol, 2-isopropyl-5-methylcyclohexyl 4-(dimethylamino)-4-oxobutyrate (2-isopropyl-5-methylcyclohexyl 4-(dimethylamino)-4-oxobutyrate); or
the agonist is TRPA1 agonist, the TRPA1 agonist comprises one or more of the following substances:
cinnamaldehyde, mustard oil, allicin, capsaicin ester, carvacrol, eugenol, piperine, gingerol, wintergreen oil, clove oil, caryophyllene, caryophyllene oxide, citral or menthol; or
the agonist is TRPV1 agonist, the TRPV1 agonist is selected from one or more of capsaicin, resiniferatoxin, vitamin D, piperine, evodiamine, scutellaria baicalenol, capsaicin ester, 6-shogaol, gingerol, gingerone, gingerol or polygonal.

2. A method comprising administering an effective amount of one or more substances in an oral spray or mouthwash for masking bitterness, wherein:
the one or more substances are a Class A substance, a Class B substance, or a combination of Class A substance and Class B substance;
the Class A substance is one or more TRPM8 agonists; and the Class B substance is one or more of TRPA1 agonists or TRPV1-4 agonists.

3. The method of claim 2, wherein:
the one or more substances are the Class A substance, and a volume concentration of the Class A substance in the oral spray or mouthwash is 0.001%-0.5%; or
the one or more substances are the Class B substance, and a volume concentration of the Class B substance in the oral spray or mouthwash is 0.001%-0.5%; or
the one or more substances are a combination of Class A substance and Class B substance, and a volume concentration of the Class A substance in the oral spray or mouthwash is 0.001%-0.5%, and a volume concentration of the Class B substance in the oral spray or mouthwash is 0.001%-0.5%.

4. The method of claim 2, wherein:
the oral spray or mouthwash reaches a peak of the bitterness masking effect within 5-10 seconds after being sprayed in an oral cavity, and the effect lasts for 30-120 seconds.

5. An oral spray or taste masking agent for masking bitterness, wherein:
the oral spray or taste masking agent comprises a Class A substance, a Class B substance, or a combination of Class A substance and Class B substance;
the Class A substance is one or more of TRPM8 agonists; and the Class B substance is one or more of TRPA1 agonists or TRPV1-4 agonists.

6. The oral spray or taste masking agent for masking bitterness according to claim 5, wherein:
the oral spray or taste masking agent is used for drugs, and the drugs comprise any one of anti-tumor drugs, drugs for treating hyperlipidemia, drugs for treating hyperglycemia, analgesics, antibiotics, antipyretics or traditional Chinese medicines, and the dosage form of the drugs comprises any one of tablet, decoction, dispersion, wine preparation, granule, mouthwash, liquid oral preparation or aerosol; or
the oral spray or taste masking agent is used for food, and the food comprises any one of ice cream, custard, cream, beverage or cake; or
the oral spray or taste masking agent is used for oral care products, and the oral care products comprise any one of toothpaste, mouthwash, chewing gum, breath freshener or mouthwash agent.

7. The oral spray or taste masking agent for masking bitterness according to claim 6, wherein:
the tablet comprises any one of a multilayer tablet, a chewable tablet, a soluble tablet, an effervescent tablet, an orodisintegrating tablet, a sustained-release tablet, a controlled-release tablet, a dispersible tablet, a lozenge, a sublingual tablet or an oral patch.

8. The method according to claim 1, wherein the substance for masking bitterness is administered before the administration of a bitter substance.
